(19) United States
(12) Patent Application Publication
(10) Pub. No.: US 2026/0034033 A1
(43) Pub. Date: Feb. 5, 2026

(54) MAKEUP COMPOSITION

(71) Applicant: L’ORÉAL, Paris (FR)

(72) Inventor: Donald KRONENBERG, Amityville, NY (US)

(73) Assignee: L’ORÉAL, Paris (FR)

(21) Appl. No.: 18/788,511

(22) Filed: Jul. 30, 2024

(51) Int. Cl. A61K 8/06; A61K 8/25; A61K 8/31; A61K 8/891; A61Q 1/02

(52) U.S. Cl. CPC A61K 8/064; A61K 8/25; A61K 8/31; A61K 8/891; A61Q 1/02; A61K 2800/30; A61K 2800/432; A61K 2800/884

(57) ABSTRACT

A makeup composition, which may be used, e.g., as a primer or setter for another composition, may be an emulsion having an aqueous discrete phase, a fatty continuous phase, and 10%-20% by weight of silica. The fatty phase may include an alkane present in an amount of 5%-30% by weight of the makeup composition. The makeup composition may be a solid emulsion. The makeup composition may be free of UV-absorbing composite particles. The makeup composition may be free of supramolecular polymers.

### TECHNICAL FIELD

The present disclosure is drawn to makeup compositions, such as, e.g., facial makeup compositions that can be worn under or over another makeup composition while remaining transparent on skin.

### BACKGROUND

A person's makeup routine may include combining various products to achieve a desired result, but the process often involves making compromises on various desirable attributes. For example, a primer might provide desirable blurring, but may not be transparent on the skin, or may not feel lightweight. A setting product might enhance the wear of underlying makeup, but might compromise its oil-absorbing ability.

Furthermore, the formulation of environmentally-friendly cosmetic products, which are designed and developed considering environmental issues, is becoming a major goal in an effort to meet global challenges. It is therefore essential to propose more sustainable compositions, preparation processes and ingredients to address these environmental concerns.

In this context, it is important to develop new cosmetic compositions with a better carbon footprint, particularly by promoting the use of renewable raw materials and/or materials with a good index of naturalness and/or materials of natural origin and, more particularly, materials of plant origin while reducing the use of compounds of petrochemical origin.

### BRIEF SUMMARY

In various aspects, a makeup composition may be provided. The makeup composition may be an emulsion, such as a solid emulsion. The makeup composition may include an aqueous discrete phase. The makeup composition may include a fatty continuous phase. The fatty continuous phase may include an alkane, such as a C15-C19 alkane. The alkane may be present in an amount of 5-30% by weight of the makeup composition. The makeup composition make include silica in an amount of 10% to 20% by weight of the makeup composition. The makeup composition may be free of ultraviolet (UV)-absorbing composite particles. The makeup composition may be free of supramolecular polymers.

In certain aspects, the aqueous discrete phase may contain more water, by weight of the makeup composition, than the amount of both silica and alkane, separately. In certain aspects, the total amount of water in the formula, by weight of the makeup composition, may be greater than a total amount of silica and the alkane combined. In certain aspects, a total amount of water in the formula may be 30% to 40% by weight of the makeup composition.

In certain aspects, the makeup composition may include a silicone emulsifier. In various aspects, the makeup composition may include a silicone oil. In certain aspects, the makeup composition may include a wax in a total amount of 2% to 5% by weight of the makeup composition. In certain aspects, the makeup composition may include a pigment. In certain aspects, the makeup composition may include a polyol. In certain aspects, the makeup composition may include cellulose and/or a cellulose derivative. In certain aspects, the makeup composition may be free of organic UV filters.

In various aspects, a method for providing improved skin aesthetics may be provided. The method may include providing a first makeup composition as disclosed herein and applying the first makeup composition to a user's face. The method may include applying a second makeup composition over the first makeup composition. Alternatively, the method may include applying a second makeup composition to the user's face prior to the first makeup composition being applied, where the first makeup composition is applied over the second makeup composition. The method may include removing the first makeup composition after a period of time, the period of time being a time between 1 hour and 48 hours.

### DETAILED DESCRIPTION

As used herein, articles such as “a” and “an” when used in a claim, are understood to mean one or more of what is claimed or described.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the term “alkane” refers to a saturated linear or branched compound formed solely from carbon and hydrogen atoms.

As used herein, the term “emulsion” refers to a composition containing a discrete phase dispersed in a continuous phase, such as a water-in-oil emulsion, the whole being homogeneous to the naked eye. As used herein, the term “solid emulsion” refers to an emulsion which does not flow under its own weight at room temperature and atmospheric pressure.

As used herein, the term “free” or “completely free of (a component)” refers to compositions that do not contain the component in any measurable degree by standard means. As used herein, the term “substantially free of (a component)” refers to compositions containing no appreciable amount of the component, for example, no more than about 1% by weight, or no more than about 0.5% by weight, or no more than about 0.3% by weight, such as no more than about 0.1% by weight, based on the weight of the composition.

As used herein, the term “oil” generally refers to a non-aqueous compound, non-miscible in water, liquid at 25° C. and atmospheric pressure (760 mmHg; 1.013.10 5 Pa). As used herein, the term “volatile oil” generally refers to an oil that is capable of evaporating on contact with the skin in less than one hour, at room temperature and atmospheric pressure. The volatile oil(s) are volatile cosmetic oils, which are liquid at room temperature, having a non-zero vapor pressure at room temperature and atmospheric pressure, ranging in particular from 0.13 Pa to 40 000 Pa (10-3 to 300 mmHg), in particular ranging from 1.3 Pa to 13 000 Pa (0.01 to 100 mmHg) and more particularly ranging from 1.3 Pa to 1300 Pa (0.01 to 10 mmHg). As used herein, the term “hydrocarbon-based oil” refers to an oil mainly containing carbon and hydrogen atoms and possibly one or more functions chosen from hydroxyl, ester, ether and carboxylic functions. As used herein, the term “silicone oil” refers to an oil comprising at least one silicon atom, and notably at least one Si—O group, and more particularly an organopolysiloxane. As used herein, the term “phenylated silicone oil” refers to a silicone oil having at least one phenyl substituent.

As used herein, the term “pigment” refers to particles that give color to keratin materials. Their solubility in water at 25° C. and atmospheric pressure (760 mmHg) is less than 0.05% by weight, preferably less than 0.01%. The term “inorganic pigment” refers to any pigment that satisfies the definition in Ullmann's encyclopedia in the chapter on inorganic pigments. The term “organic pigment” refers to any pigment that satisfies the definition in Ullmann's encyclopedia in the chapter on organic pigments.

As used herein, the term “wax” generally refers to a lipophilic compound that is solid at ambient temperature (25° C.), with a reversible solid/liquid change in state, having a melting point of greater than or equal to about 30° C., which may be up to 200° C. and in particular up to 120° C. As used herein, the term “soft wax” refers to waxes which have a melting point of below about 65° C. As used herein, the term “hard wax” refers to waxes other than soft waxes—that is, waxes which have a melting point of equal to or greater than about 65° C. As used herein, the term “silicone wax” refers to a wax comprising at least one silicon atom, and notably at least one Si—O group.

Unless otherwise noted, all usage concentrations of compounds listed herein refer to the active amount of the compound as a percent by weight of the makeup composition.

The present disclosure is drawn to a facial makeup product, such as a primer and/or setter, that provides a lightweight, fresh sensation while also providing blurring, oil control, and enhancing the wear of makeup applied under or over the product while also remaining transparent (e.g., no white cast) on the skin.

The disclosed makeup composition may be an emulsion. The emulsion is preferably in the form of a solid emulsion. The makeup composition is preferably in the form of a water-in-oil phase. Thus, the makeup composition may include an aqueous discrete phase and a fatty continuous phase. The fatty continuous phase includes an alkane, such as a C15-C19 alkane. The alkane is present in an amount of 5-30% by weight of the makeup composition. In addition, the composition may include particulates, which may be dispersed in the composition, such as within the fatty continuous phase. The particulates include silica at 10%-20% by weight of the makeup composition.

### Alkane

The fatty continuous phase includes an alkane. The alkane is preferably a C15-C19 alkane.

There may be a single alkane. There may be a plurality of alkanes. The alkane may be a volatile oil. The alkane may be a non-volatile oil. The alkane may be a wax. Each alkane may be an alkane having a carbon chain length of 8 to 30 carbons. Each alkane may be an alkane having a carbon chain length of 10 to 25 carbons. Each alkane may be an alkane having a carbon chain length of 15 to 19 carbons.

The alkane may be present in a total amount of 5% to 30% by weight of the makeup composition. The alkane may be present in a total amount of at least 5%, at least 6%, at least 7%, at least 8%, at least 9%, or at least 10% by weight of the makeup composition. The alkane may be present in a total amount of no more than 30%, no more than 29%, no more than 28%, no more than 27%, no more than 26%, no more than 25%, no more than 24%, no more than 23%, no more than 22%, no more than 21%, or no more than 20% by weight of the makeup composition. In a preferred embodiment, the alkane may be present in a total amount of 10% to 20% by weight of the makeup composition.

### Silica

The makeup composition may include silica. The silica may include only a single silica. The silica may include a plurality of silicas.

The silica may be a silica aerogel. The silica aerogel may be a hydrophobic silica aerogel, preferably of silylated silica (INCI name: silica silylate).

The term “hydrophobic silica” is understood to mean any silica whose surface is treated with silylating agents, for example with halogenated silanes, such as alkylchlorosilanes, siloxanes, in particular dimethylsiloxanes, such as hexamethyldisiloxane, or silazanes, so as to functionalize the OH groups with silyl groups Si—Rn, for example trimethylsilyl groups.

The silica may be a hydrophobic silica modified at the surface with trimethylsilyl groups (trimethylsiloxylated silica). The silica may be hydrophobic silica aerogel sold under the name VM-2260 (INCI name: silica silylate) by Dow Corning, the particles of which exhibit an average size of approximately 1000 microns and a specific surface per unit of weight ranging from 600 to 800 m²/g, or VM-2270 (INCI name: silica silylate) by Dow Corning, the particles of which exhibit an average size ranging from 5 to 15 microns and a specific surface per unit of weight ranging from 600 to 800 m²/g. The silica may be aerogels sold by Cabot under the names Aerogel TLD 201, Aerogel OGD 201 and Aerogel TLD 203.

The makeup composition make include silica in an amount of 10% to 20% by weight of the makeup composition.

### Water

The composition includes water. In some embodiments, the total amount of water may be at least 20% of the makeup composition. The total amount of water may be at least 20%, at least 25%, or at least 30% of the makeup composition. The total amount of water may be no more than 50%, no more than 45%, or no more than 40%, of the makeup composition. In preferred embodiments, the total amount of water in the formula may be 30% to 40% by weight of the makeup composition.

In certain aspects, the aqueous discrete phase may contain more water, by weight of the makeup composition, than the amount of both silica and alkane, separately. For example, the makeup composition may include 20% alkane, 15% silica, and 30% water (30%>20% and 30%>15%).

In certain aspects, the total amount of water in the formula, by weight of the makeup composition, may be greater than a total amount of silica and the alkane combined. For example, the makeup composition may include 14% silica, 16% alkane, and 35% water (35%>14%+16%). Other Components

### Emulsifiers

The makeup composition may include an emulsifier. In some embodiments, the emulsifier may be an anionic, non-ionic emulsifier, or a combination thereof. In certain embodiments, useful emulsifiers for the composition of the present invention are those with an HLB (hydrophilic-lipophilic balance) no greater than 7. Although any cosmetically acceptable emulsifier may be used in the compositions, excellent results can be obtained using silicone-based emulsifiers (sometimes referred to herein as “silicone emulsifier”).

In some embodiments, the makeup composition may be substantially free of a cationic surfactant. In some embodiments, the makeup composition may be free of a cationic surfactant. In some embodiments, the makeup composition may be substantially free of an anionic surfactant. In some embodiments, the makeup composition may be free of an anionic surfactant. In some embodiments, the makeup composition may be substantially free of a non-ionic surfactant. In some embodiments, the makeup composition may be free of a non-ionic surfactant.

### Silicone Emulsifier

The makeup composition may include a silicone emulsifier. Non-limiting examples of some useful silicone emulsifiers are: (1) dimethicone and PEG/PPG-18/18 dimethicone (e.g., X-22-6711D from Shin-Etsu); (2) dimethicone and dimethicone crosspolymer (e.g., Dow Corning 9041 Silicone Elastomer Blend); (3) dimethicone and dimethicone/PEG-10/15 crosspolymer (KSG-210 from Shin-Etsu); and (4) dimethicone and dimethicone/polyglycerin-3 crosspolymer (KSG 710 from Shin-Etsu).

In certain embodiments, the silicone emulsifier that may be used in the present invention may be chosen from polyether substituted linear or branched polysiloxane copolymers. Non-limiting examples of such emulsifiers include PEG-10 dimethicone available under the name of ES-5612 from Dow Corning Corporation, or under the name of KF-6017 from Shin-Etsu; dimethicone (and) PEG/PPG-18/18 dimethicone available under the tradename of ES-5226 DM from Dow Corning Corporation; PEG-9 polydimethylsiloxyethyl dimethicone available under the name KF-6028 from Shin-Etsu; and/or lauryl PEG-9 polydimethylsiloxyethyl dimethicone available under the name KF-6038 from Shin-Etsu. A preferred embodiment utilizes PEG-9 polydimethylsiloxyethyl dimethicone.

The silicone emulsifier may be present in an amount ranging from about 0.1%, 1%, 3%, or 5% to about 6%, 8%, or 10% by weight of the makeup composition, including all ranges and subranges there between. In some embodiments, the makeup composition may be substantially free of a silicone emulsifier. In some embodiments, the makeup composition may be free of a silicone emulsifier.

### Non-Silicone Emulsifier

The makeup composition may include a non-silicone emulsifier.

Non-limiting examples of non-silicone emulsifier include alkyl polyglucosides; alcohols, alpha-diols, alkylphenols and esters of fatty acids, being ethoxylated, propoxylated or glycerolated; ethoxylated fatty esters; glyceryl esters of fatty acids; fatty alcohol ethoxylates; alkyl phenol ethoxylates; fatty acid alkoxylates; and mixtures thereof. In some instances, nonionic surfactants such as polyoxyethylenated C8-C30 fatty acid esters of sorbitan may be used. In some embodiments, the composition may include polyglyceryl-4 isostearate. In some embodiments, the composition may include polysorbate 20 (Tween 20), glyceryl stearate, stearic acid, and/or sodium dihydroxycetyl phosphate.

The non-silicone emulsifier may be present in an amount ranging from about 0.1%, 1%, 3%, or 5% to about 6%, 8%, or 10% by weight of the makeup composition, including all ranges and subranges there between. In some embodiments, the makeup composition may be substantially free of a non-silicone emulsifier. In some embodiments, the makeup composition may be free of a non-silicone emulsifier.

### Cosmetic Oil

In addition to the alkane, the makeup composition may include a cosmetic oil. The makeup composition may include a single cosmetic oil. The makeup composition may include a plurality of cosmetic oils. The cosmetic oils may include a silicone oil and/or a non-alkane hydrocarbon oil.

The makeup composition may include 1% to 50% of a cosmetic oil. The makeup composition may include from 1%, 2%, 5%, or 10% up to 15%, 20%, 25%, 30%, 35%, 40%, 45%, or 50% of a cosmetic oil, including all ranges and subranges there between. The makeup composition may be substantially free of a cosmetic oil. The makeup composition may be free of a cosmetic oil.

### Silicone Oil

The cosmetic oil may include a silicone oil. The silicone oil may be a volatile silicone oil. The silicone oil may be a non-volatile silicone oil. The makeup composition may include a phenylated silicone oil. The silicone oil may be a non-phenylated silicone oil. The makeup composition may be free of a phenylated silicone oil. The makeup composition may be free of a non-phenylated silicone oil.

Among the non-volatile silicone oils, non-limiting examples include non-volatile polydimethylsiloxanes (PDMS); phenyl silicones such as phenyl trimethicones, phenyl dimethicones, diphenyl dimethicones, trimethylpentaphenyltrisiloxanes, tetramethyltetraphenyltrisiloxanes, trimethylsiloxyphenyl dimethicones, and diphenylsiloxyphenyl trimethicones.

The makeup composition may include 1% to 50% of a non-volatile silicone oil. The makeup composition may include from 1%, 2%, 5%, or 10% up to 15%, 20%, 25%, 30%, 35%, 40%, 45%, or 50% of a non-volatile silicone oil, including all ranges and subranges there between. The makeup composition may be substantially free of a non-volatile silicone oil. The makeup composition may be free of a non-volatile silicone oil.

In some embodiments, the volatile oil may be a linear or cyclic volatile silicone oil. Linear volatile silicone oils that may be mentioned include octamethyltrisiloxane, decamethyltetrasiloxane, dodecamethylpentasiloxane and mixtures thereof. Cyclic volatile silicone oils that may be mentioned include octamethylcyclotetrasiloxane, decamethylcyclopentasiloxane and dodecamethylcyclohexasiloxane, and mixtures thereof.

The makeup composition may include 1% to 50% of a volatile silicone oil. The makeup composition may include from 1%, 2%, 5%, or 10% up to 15%, 20%, 25%, 30%, 35%, 40%, 45%, or 50% of a volatile silicone oil, including all ranges and subranges there between. The makeup composition may be substantially free of a volatile silicone oil. The makeup composition may be free of a volatile silicone oil.

### Non-Alkane Hydrocarbon-Based Oil

The cosmetic oil may include a non-alkane hydrocarbon-based oil. The non-alkane hydrocarbon-based oil may be a volatile non-alkane hydrocarbon-based oil. The hydrocarbon-based oil may be a non-volatile non-alkane hydrocarbon-based oil.

Non-limiting examples of hydrocarbon-based oils include:
volatile linear or branched non-alkanes, such as linear or branched C8-C16 esters;
hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil.
synthetic ethers containing from 10 to 40 carbon atoms;
linear or branched hydrocarbons of mineral or synthetic origin, such as petroleum jelly, polydecenes, hydrogenated polyisobutene such as Parleam, and 40 squalane;
synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is ≥10, for instance cetearyl octanoate, isopropyl myristate, isopropyl palmitate, C12-C15 alkyl benzoate, 2-ethylphenyl benzoate, isopropyl lanolate, hexyl laurate, diisopropyl adipate, isononyl isononanoate, oleyl erucate, 2-ethylhexyl palmitate, isostearyl isostearate, diisopropyl sebacate, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C12-C13 alkyl) tartrates, such as those sold under the name COSMACOL® ETI esters by Sasol, and also di(linear C14-C15 alkyl) tartrates, such as those sold under the name COSMACOL® ETL esters by Sasol;
fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol; higher fatty acids, such as oleic acid, linoleic acid or linolenic acid;
carbonates, such as dicaprylyl carbonate, such as the product sold under the name CETIOL® CC emollient by Cognis;
fatty amides, such as isopropyl N-lauroyl sarcosinate, such as the product sold under the trade name ELDEW® SL 205 amino acid-derived emollient from Ajinomoto; and
essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

In certain embodiments, the non-alkane hydrocarbon-based oil may include glyceride triesters and in particular caprylic/capric acid triglycerides, synthetic esters and in particular isononyl isononanoate, oleyl erucate, C12-C15 alkyl benzoate, 2-ethylphenyl benzoate and fatty alcohols, such as octyldodecanol.

The makeup composition may include 1% to 50% of a non-alkane hydrocarbon-based oil. The makeup composition may include from 1%, 2%, 5%, or 10% up to 15%, 20%, 25%, 30%, 35%, 40%, 45%, or 50% of a non-alkane hydrocarbon-based oil, including all ranges and subranges there between. The makeup composition may be substantially free of a non-alkane hydrocarbon-based oil. The makeup composition may be free of a non-alkane hydrocarbon-based oil. The makeup composition may be substantially free of a volatile non-alkane hydrocarbon-based oil. The makeup composition may be free of a volatile non-alkane hydrocarbon-based oil. The makeup composition may be substantially free of a non-volatile non-alkane hydrocarbon-based oil. The makeup composition may be free of a non-volatile non-alkane hydrocarbon-based oil.

### Wax

The makeup composition may include a wax. The wax may be present in a total amount of 2% to 5% by weight of the makeup composition. The wax may be present in a total amount of 2%, 3%, or 4% up to 3%, 4%, or 5% by weight of the makeup composition, including all ranges and subranges there between. In some embodiments, the makeup composition may be substantially free of a wax.

In some embodiments, the makeup composition may include a soft wax. Non-limiting examples of soft waxes may include paraffin wax, beeswax, synthetic beeswax, and ceresin wax. In some embodiments, the makeup composition may be free of a wax. In some embodiments, the makeup composition may be substantially free of a soft wax. In some embodiments, the makeup composition may be free of a soft wax.

In some embodiments, the makeup composition may include a hard wax. Non-limiting examples of hard waxes may include carnauba wax, microcrystalline wax, synthetic wax, candelilla wax, and ozokerite. In a preferred embodiment, the wax is candelilla wax. In some embodiments, the makeup composition may be substantially free of a hard wax. In some embodiments, the makeup composition may be free of a hard wax.

In some embodiments, the makeup composition may include a silicone wax. Non-limiting examples of silicone waxes include alkyl or alkoxy dimethicones, such as (C20-C60) alkyl dimethicones, in particular (C30-C45) alkyl dimethicones, such as the silicone wax sold under the name SF-1642 by the company Momentive or the (C30-C45) alkyl dimethylsilyl polypropyl silsesquioxane under the name DOWSIL™ SW-8005 C30 Resin Wax sold by Dow Inc; and silicone waxes obtained by esterification with a (poly) alkoxylated silicone, such s silicone beeswax, silicone candelilla wax, or silicone carnauba wax. In some embodiments, the makeup composition may be substantially free of a silicone wax. In some embodiments, the makeup composition may be free of a silicone wax. In some embodiments, the makeup composition may include a non-silicone wax. In some embodiments, the makeup composition may be substantially free of a non-silicone wax. In some embodiments, the makeup composition may be free of a non-silicone wax.

### Pigment

In certain aspects, the makeup composition may include a pigment.

Examples of suitable pigments include, but are not limited to, inorganic pigments, organic pigments, lakes, pearlescent pigments, iridescent or optically variable pigments, and mixtures thereof. The pigments may be chosen from the organic and/or mineral pigments known in the art. The pigments may be in the form of powder or of pigmentary paste. They may be coated or uncoated. Said pigments may optionally be surface treated within the scope of the present invention but are not limited to treatments such as silicones, perfluorinated compounds, lecithin, and amino acids.

The pigments may be chosen, for example, from inorganic pigments, organic pigments, lakes, pigments with special effects such as nacres or glitter flakes, and mixtures thereof. Non-limiting examples of inorganic pigments include those selected from the group consisting of rutile or anatase titanium dioxide, coded in the Color Index under the reference CI 77,891; black, yellow, red and brown iron oxides, coded under references CI 77,499, 77, 492 and, 77,491; manganese violet (CI 77,742); ultramarine blue (CI 77,007); chromium oxide (CI 77,288); chromium hydrate (CI 77,289); and ferric blue (CI 77,510) and mixtures thereof.

The pigment may be an inorganic pigment. Non-limiting examples include iron oxides, chromium oxides, manganese violet, ultramarine blue, chromium hydrate, ferric blue and titanium oxide.

The pigment may be an organic pigment. Non-limiting examples include nitroso, nitro, azo, xanthene, quinoline, anthraquinone, phthalocyanin, metal-complex, isoindolinone, isoindoline, quinacridone, perinone, perylene, diketopyrrolopyrrole, thioindigo, dioxazine, triphenylmethane and quinophthalone compounds.

In particular, the white or colored organic pigments may be chosen from carmine, carbon black, aniline black, azo yellow, quinacridone, phthalocyanin blue, sorghum red, the blue pigments codified in the Color Index under the references CI 42090, 69800, 69825, 73000, 74100 and 74160, the yellow pigments codified in the Color Index under the references CI 11680, 11710, 15985, 19140, 20040, 21100, 21108, 47000 and 47005, the green pigments codified in the Color Index under the references CI 61565, 61570 and 74260, the orange pigments codified in the Color Index under the references CI 11725, 15510, 45370 and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and pigments obtained by oxidative polymerization of indole or phenolic derivatives. Non-limiting examples of organic pigments and lakes include, but are not limited to, D&C Red No. 19 (CI 45,170), D&C Red No. 9 (CI 15,585), D&C Red No. 21 (CI 45,380), D&C Orange No. 4 (CI 15,510), D&C Orange No. 5 (CI 45,370), D&C Red No. 27 (CI 45,410), D&C Red No. 13 (CI 15,630), D&C Red No. 7 (CI 15,850), D&C Red No. 6 (CI 15,850), D&C Yellow No. 5 (CI 19,140), D&C Red No. 36 (CI 12,085), D&C Orange No. 10 (CI 45,425), D&C Yellow No. 6 (CI 15,985), D&C Red No. 30 (CI 73,360), D&C Red No. 3 (CI 45,430) and the dye or lakes based on cochineal carmine (CI 75,570) and mixtures thereof.

The pigments in accordance with the invention may also be in the form of composite pigments. These composite pigments may be compounds especially of particles comprising a inorganic core, at least one binder for ensuring the binding of the organic pigments to the core, and at least one organic pigment at least partially covering the core.

The organic pigment may also be a lake. The term “lake” refers dyes adsorbed onto insoluble particles, the assembly thus obtained remaining insoluble during use.

The inorganic substrates onto which the dyes are adsorbed are, for example, alumina, silica, calcium sodium borosilicate or calcium aluminum borosilicate, and aluminum.

Among the dyes, mention may be made of cochineal carmine. Non-limiting examples of dyes include: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 10 (CI 77 002), D&C Green 3 (CI 42 053), D&C Blue 1 (CI 42 090). An example of a lake is D&C Red 7 (CI 15 850:1).

The pigment may also be a pigment with special effects. The term “pigments with special effects” refers pigments that generally create a non-uniform colored appearance (characterized by a certain shade, a certain vivacity and a certain lightness) that changes as a function of the conditions of observation (light, temperature, observation angles, etc.). They thus contrast with colored pigments that afford a standard uniform opaque, semi-transparent or transparent shade.

Several types of pigment with special effects exist: those with a low refractive index, such as fluorescent, photochromic or thermochromic pigments, and those with a higher refractive index, such as nacres or glitter flakes.

Examples of pigments with special effects that may be mentioned include nacreous pigments such as titanium mica coated with an iron oxide, mica coated with an iron oxide, mica coated with bismuth oxychloride, titanium mica coated with chromium oxide, titanium mica coated with an organic dye especially of the abovementioned type, and also nacreous pigments based on bismuth oxychloride. They may also be mica particles at the surface of which are superposed at least two successive layers of metal oxides and/or of organic dyestuffs.

The nacres may more particularly have a yellow, pink, red, bronze, orange, brown, gold and/or coppery color or tint.

Non-limiting examples of nacres include gold-colored nacres sold especially by the company Engelhard under the name Gold 222C (a CLOISONNÉ® pigment), Sparkle gold (a TIMICA® pigment), Gold 4504 (a CHROMA-LITE® pigment) and Monarch gold 233X (a CLOISONNÉ® pigment); the bronze nacres sold especially by the company Merck under the name Bronze fine (17384) (a COLORONA® pigment) and Bronze (17353) (a COLORONA® pigment), by the company Eckart under the name PRESTIGE™ Bronze pigment and by the company Engelhard under the name Super bronze (a CLOISONNE® pigment); the orange nacres sold especially by the company Engelhard under the name Orange 363C (a CLOISONNE® pigment) and Orange MCR 101 and by the company Merck under the name Passion orange (a COLORONA® pigment) and Matte orange (17449); the brown nacres sold especially by the company Engelhard under the name Nu-antique copper 340XB (a CLOISONNE® pigment) and Brown CL4509 (a CHROMA-LITE® pigment); the nacres with a copper tint sold especially by the company Engelhard under the name Copper 340A (a TIMICA® pigment) and by the company Eckart under the name Prestige Copper; the nacres with a red tint sold especially by the company Merck under the name Sienna fine (17386) (a COLORONA® pigment); the nacres with a yellow tint sold especially by the company Engelhard under the name Yellow (4502) (a CHROMA-LITE® pigment); the red nacres with a gold tint sold especially by the company Engelhard under the name Sunstone G012 (a GEMTONE® pigment); the black nacres with a gold tint sold especially by the company Engelhard under the name Nu antique bronze 240 AB (a TIMICA® pigment), the blue nacres sold especially by the company Merck under the name Matte blue (17433), Dark Blue (117324) (a COLORONA® pigment), the white nacres with a silvery tint sold especially by the company Merck under the name XIRONA® Silver pigment, and the golden-green pink-orange nacres sold especially by the company Merck under the name Indian summer (a XIRONA® pigment), and mixtures thereof.

In addition to nacres on a mica support, multilayer pigments based on synthetic substrates such as alumina, silica, sodium calcium borosilicate or calcium aluminum borosilicate, and aluminum, may be envisaged.

Non-limiting examples of pearlescent pigments include those selected from the group consisting of the white pearlescent pigments such as mica coated with titanium oxide, mica coated with titanium dioxide, bismuth oxychloride, titanium oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof.

Mention may also be made of pigments with an interference effect that are not fixed onto a substrate, for instance liquid crystals or holographic interference glitter flakes. Pigments with special effects also comprise fluorescent pigments, whether these are substances that are fluorescent in daylight or that produce an ultraviolet fluorescence, phosphorescent pigments, photochromic pigments, thermochromic pigments and quantum dots.

The variety of pigments that may be used in the present invention makes it possible to obtain a wide range of colors, and also particular optical effects such as metallic effects or interference effects.

The effective diameter of the pigment particles may generally be between 10 nm and 200 μm, such as between 20 nm and 80 μm, such as between 30 nm and 50 μm.

In some embodiments, the pigment may be present in a total amount of no more than 30% by weight of the makeup composition. In some embodiments, the pigment may be present in a total amount of no more than 20% by weight of the makeup composition. The pigment may be present in a total amount of no more than 15% by weight of the makeup composition. The pigment may be present in a total amount of no more than 10% by weight of the makeup composition. The pigment may be present in a total amount of no more than 8% by weight of the makeup composition. The pigment may be present in a total amount of no more than 6% by weight of the makeup composition. The pigment may be present in a total amount of no more than 5% by weight of the makeup composition. The pigment may be present in a total amount of no more than 4% by weight of the makeup composition. The pigment may be present in a total amount of no more than 3% by weight of the makeup composition. The pigment may be present in a total amount of at least 0.1% by weight of the makeup composition. The pigment may be present in a total amount of at least 1% by weight of the makeup composition. The pigment may be present in a total amount of at least 2% by weight of the makeup composition. The pigment may be present in a total amount of at least 3% by weight of the makeup composition. The pigment may be present in a total amount of at least 4% by weight of the makeup composition. The pigment may be present in a total amount of at least 5% by weight of the makeup composition. The pigment may be present in a total amount of at least 10% by weight of the makeup composition. The pigment may be present in a total amount of at least 15% by weight of the makeup composition. The pigment may be present in a total amount of at least 20% by weight of the makeup composition. In some embodiments, the pigment may be present in a total amount of 10-20% by weight of the makeup composition.

In some embodiments, the composition may be substantially free of a pigment. In some embodiments, the composition may be free of a pigment. In some embodiments, the composition may be substantially free of titanium dioxide. In some embodiments, the composition may be free of titanium dioxide.

### Polyol

In certain aspects, the makeup composition may include a polyol. The polyol may be present in a total amount of no more than 20% by weight of the makeup composition. The polyol may be present in a total amount of no more than 15% by weight of the makeup composition. The polyol may be present in a total amount of no more than 10% by weight of the makeup composition. The polyol may be present in a total amount of no more than 5% by weight of the makeup composition. The polyol may be present in a total amount of at least 1% by weight of the makeup composition. The polyol may be present in a total amount of at least 2% by weight of the makeup composition. The polyol may be present in a total amount of at least 3% by weight of the makeup composition. The polyol may be present in a total amount of at least 4% by weight of the makeup composition. The polyol may be present in a total amount of at least 5% by weight of the makeup composition. The polyol may be present in a total amount of at least 6% by weight of the makeup composition. The polyol may be present in a total amount of at least 7% by weight of the makeup composition. The polyol may be present in a total amount of at least 8% by weight of the makeup composition. The polyol may be present in a total amount of at least 9% by weight of the makeup composition. The polyol may be present in a total amount of at least 10% by weight of the makeup composition. The makeup composition may be substantially free of a polyol. The makeup composition may be free of a polyol.

The polyol may be a polyhydric alcohol. Non-limiting examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, hexylene glycol (2-methyl-2,4-pentanediol), tetraethylene glycol, 1,6-hexanediol, caprylyl glycol (1,2-octanediol), polyethylene glycol, 1,2,4-butanetriol, 1,2,6-hexanetriol, and a mixture thereof. In some embodiments, the composition may be substantially free of a polyhydric alcohol. In some embodiments, the composition may be free of a polyhydric alcohol.

The polyol may be an aliphatic diol. Non-limiting examples of aliphatic diols include 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol. In some embodiments, the composition may be substantially free of an aliphatic diol. In some embodiments, the composition may be free of an aliphatic diol.

The polyol may be an alkyl dimethicone copolyols such as lauryl PEG/PPG-18/18 methicone, cetyl PEG/PPG-10/1 dimethicone, and/or cetyl PEG/PPG-10/1 dimethicone. In some embodiments, the composition may be substantially free of alkyl dimethicone copolyols. In some embodiments, the composition may be free of alkyl dimethicone copolyols.

The polyol may be a monosaccharide polyol, such as mannitol, erythritol, xylitol, and/or sorbitol. In some embodiments, the composition may be substantially free of monosaccharide polyols. In some embodiments, the composition may be free of monosaccharide polyols.

### Polysaccharides

In certain aspects, the makeup composition may include a polysaccharide.

The polysaccharide may be present in a total amount of no more than 10% by weight of the makeup composition. The polysaccharide may be present in a total amount of no more than 9% by weight of the makeup composition. The polysaccharide may be present in a total amount of no more than 8% by weight of the makeup composition. The polysaccharide may be present in a total amount of no more than 7% by weight of the makeup composition. The polysaccharide may be present in a total amount of no more than 6% by weight of the makeup composition. The polysaccharide may be present in a total amount of no more than 5% by weight of the makeup composition. The polysaccharide may be present in a total amount of no more than 4% by weight of the makeup composition. The polysaccharide may be present in a total amount of no more than 3% by weight of the makeup composition. The polysaccharide may be present in a total amount of at least 1% by weight of the makeup composition. The polysaccharide may be present in a total amount of at least 2% by weight of the makeup composition. The polysaccharide may be present in a total amount of at least 3% by weight of the makeup composition. The polysaccharide may be present in a total amount of at least 4% by weight of the makeup composition. The polysaccharide may be present in a total amount of at least 5% by weight of the makeup composition. The makeup composition may be substantially free of a polysaccharide. The makeup composition may be free of a polysaccharide. In some embodiments, the polysaccharide may be hyaluronic acid, or a salt thereof such as the sodium salt (sodium hyaluronate).

The polysaccharide may be a glycosaminoglycans (GAG) such as hyaluronic acid, chondroitin sulfate, dermatan sulfate, keratan sulfate, and preferably hyaluronic acid; xylans (or arabinoxylans) and derivatives.

The polysaccharide may be cellulose and/or a cellulose derivative. The composition may be substantially free of cellulose and/or a cellulose derivative. The composition may be free of cellulose and/or a cellulose derivative.

The term “cellulose derivative” means a compound comprising at least one cellobiose unit having the following structure:

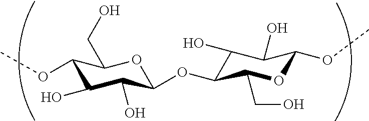

in which one or more hydroxyl groups may be substituted.

The cellulose derivative may be a water-soluble cellulose derivative. The term “water-soluble” means soluble in water, in particular in a proportion of at least 10 grams per litre of water, preferably at least 20 g/l, better still at least 50 g/l, at a temperature of less than or equal to 35° C. Non-limiting examples of water-soluble cellulose derivatives include cationic cellulose polymers such as cellulose copolymers and cellulose derivatives grafted with a water-soluble monomer of quaternary ammonium. Such derivatives include, e.g., hydroxyalkylcelluloses, for instance, hydroxymethyl-, hydroxyethyl-, and hydroxypropylcelluloses grafted, for example, with a salt chosen from methacryloylethyltrimethylammonium, methacrylamidopropyltrimethylammonium, and dimethyldiallylammonium salts. In some embodiments, the composition may be substantially free of a water-soluble cellulose derivative. In some embodiments, the composition may be free of a water-soluble cellulose derivative.

The cellulose derivative may be a water-insoluble cellulose derivative. Non-limiting examples of water-insoluble cellulose derivatives include nitrocellulose, cellulose acetate, cellulose acetate phthalate, hydroxypropylmethylcellulose phthalate. In some embodiments, the composition may be substantially free of a water-insoluble cellulose derivative. In some embodiments, the composition may be free of a water-insoluble cellulose derivativ.

### Ultraviolet (UV) Filters

The composition may include a UV filter.

The UV filter may be present in a total amount of no more than 20% by weight of the composition. The UV filter may be present in a total amount of no more than 15% by weight of the composition. The UV filter may be present in a total amount of no more than 10% by weight of the composition. The UV filter may be present in a total amount of no more than 5% by weight of the composition. The UV filter may be present in a total amount of at least 1% by weight of the composition. The UV filter may be present in a total amount of at least 2% by weight of the composition. The UV filter may be present in a total amount of at least 3% by weight of the composition. The UV filter may be present in a total amount of at least 4% by weight of the composition. The UV filter may be present in a total amount of at least 5% by weight of the composition. The makeup composition may be substantially free of a UV filter. The makeup composition may be free of a UV filter.

The UV filter may be a mineral UV filter. Non-limiting examples of mineral UV filters includes UV-grade titanium dioxide and zinc oxide. In some embodiments, the composition may be substantially free of a mineral UV filter. In some embodiments, the composition may be free of a mineral UV filter.

The makeup composition should be free of UV-absorbing composite particles. Such UV-absorbing composite particles may be a composite particle as described in, e.g., U.S. Pat. No. 9,913,782, and generally include a (i) matrix comprising at least one organic material and/or at least one mineral material, and (ii) a UV filter, such as an organic UV filter or a mineral UV filter such as titanium dioxide, the mineral UV filters optionally being treated with a surface agent. Such composite particles typically have larger particle sizes, such as 0.1 μm or greater. Such composite particles may include materials where multiple metal oxide particles are disposed within a matrix. Such composite particles may also include, e.g., core-shell particles, often having an organic shell around a metal oxide core.

The mineral UV filter may be in free form. The term “free form” means that the mineral UV filter is not incorporated into a matrix and/or onto the surface of the matrix of a composite particle.

The UV filter may be an organic UV filter. Non-limiting examples of organic UV filters include, e.g., homosalate, ethylhexyl salicylate, and octocrylene. In some embodiments, the composition may be substantially free of an organic UV filter. In some embodiments, the composition may be free of an organic UV filter.

### Preservative

The makeup composition may include a preservative.

Representative examples of preservatives include alkyl para-hydroxybenzoates, wherein the alkyl radical has from 1, 2, 3, 4, 5 or 6 carbon atoms and preferably from 1 to 4 carbon atoms e.g., methyl para-hydroxybenzoate(methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben), and phenoxyethanol.

In some embodiments, the preservative may be present in a total amount of no more than 15% by weight of the composition. In some embodiments, the preservative may be present in a total amount of no more than 10% by weight of the composition. In some embodiments, the preservative may be present in a total amount of no more than 5% by weight of the composition. In some embodiments, the makeup composition may be present in a total amount of at least 1% by weight of the composition. In some embodiments, the makeup composition may be substantially free of a preservative. In some embodiments, the makeup composition may be free of a preservative.

### Fragrance

The makeup composition may include a fragrance. In some embodiments, the composition may be substantially free of a fragrance. In some embodiments, the composition may be free of a fragrance.

### Conditioning Agents

The makeup composition may include a conditioning agent, such as an amphoteric conditioning agent. In some embodiments, the composition may be substantially free of a conditioning agent. In some embodiments, the composition may be free of a conditioning agent.

Non-limiting examples of amphoteric conditioning agents include, e.g., arginine, asparagines, aspartic acid, glycine, glutamic acid, lysine, methionine, phenylalanine, serine, threonine, tyrosine, tryptophan, valine, gelatin, Quaternium-27, oleamidopropyl betaine, disodium cocoamphodiacetate, disodium cocoamphodipropionate, disodium lauroamphodiacetate, sodium cocoamphopropionate, sodium cocoamphoacetate, meadowfoam delta lactone, cocoamidopropyl betaine, cocoamidopropyl hydroxysultaine, lauramidopropyl betaine, carnitine, hydroxyproline, acetyl hydroxy proline, isoleucine, lauroyl lysine, lauroyl sarcosine, polylysine, proline, rice amino acids, silk amino acids, wheat amino acids and mixtures thereof.

### Antioxidants

The makeup composition may include an antioxidant. In some embodiments, an antioxidant may be present in the composition in a total amount of 0-0.5% by weight of the composition. In some embodiments, the composition may be substantially free of an antioxidant. In some embodiments, the composition may be free of an antioxidant.

Non-limiting examples of antioxidants include, e.g., tocopherol, pentaerythrityl tetra-di-t-butyl hydroxyhydrocinnamate, ascorbic acid, ascorbyl compounds such as ascorbyl dipalmitate, t-butylhydroquinone, polyphenols such as phloroglucinol, sodium sulfite, and erythorbic acid.

### Ceramides

The makeup composition may include a ceramide. In some embodiments, a ceramide may be present in the composition in a total amount of 0-0.2% by weight of the composition. In some embodiments, the composition may be substantially free of a ceramide. In some embodiments, the composition may be free of a ceramide.

Ceramides are a family of waxy lipid molecules that are composed of sphingosine and a fatty acid. Ceramides include ceramide 1, ceramide 2, ceramide 3, ceramide 4, ceramide 5, ceramide 1A, ceramide 6 II, ceramide AP, ceramide EOP, ceramide EOS, ceramide NP, ceramide NG, ceramide NS, ceramide AS, and ceramide NS dilaurate. In some instances, the cosmetic composition may include ceramide NP.

### Moisturizers and Humectants

The makeup composition may include an additional moisturizer and/or humectant. In some embodiments, an additional moisturizer and/or humectant may be present in the composition in a total amount of 0-0.2% by weight of the composition. In some embodiments, the composition may be substantially free of an additional moisturizer and/or humectant. In some embodiments, the composition may be free of an additional moisturizer and/or humectant.

Moisturizers or humectants that may especially be mentioned include sorbitol, polyhydric alcohols, preferably of C2-C8 and more preferably C3-C6, preferably such as glycerol, propylene glycol, tripropylene glycol, 1,3-butylene glycol, dipropylene glycol and diglycerol, and mixtures thereof, glycerol and derivatives thereof, urea and derivatives thereof, especially Hydrovance® (2-hydroxyethylurea) sold by National Starch, lactic acids, hyaluronic acid, AHAs, BHAs, sodium pidolate, xylitol, serine, sodium lactate, ectoin and derivatives thereof, chitosan and derivatives thereof, collagen, plankton, an extract of Imperata cylindra sold under the name Moist 24® by the company Sederma, acrylic acid homopolymers, for instance Lipidure-HM® from NOF Corporation, β-glucan and in particular sodium carboxymethyl β-glucan from Mibelle-AG-Biochemistry; a mixture of passionflower oil, apricot oil, corn oil and rice bran oil sold by Nestle under the name NutraLipids®; a C-glycoside derivative such as those described in patent application WO 02/051 828 and in particular C-β-D-xylopyranoside-2-hydroxypropane in the form of a solution containing 30% by weight of active material in a water/propylene glycol mixture (60/40% by weight) such as the product manufactured by Chimex under the trade name Mexoryl SBB®; an oil of musk rose sold by Nestle; an extract of the microalga Prophyridium cruentum enriched with zinc, sold by Vincience under the name Algualane Zinc®; spheres of collagen and of chondroitin sulfate of marine origin (Ateocollagen) sold by the company Engelhard Lyon under the name Marine Filling Spheres; hyaluronic acid spheres such as those sold by the company Engelhard Lyon; and arginine.

### Calmatives

The makeup composition may include a calmative (e.g., a compound that reduces the sensation of stinging, itching, or tautness of the skin). In some embodiments, a calmative may be present in the composition in a total amount of 0-0.2% by weight of the composition. In some embodiments, the composition may be substantially free of a calmative. In some embodiments, the composition may be free of a calmative.

Non-limiting examples of calmatives include, e.g., procyannidol oligomers, vitamins E, C, B5 and B3, caffeine and derivatives thereof, pentacylic triterpenes and plant extracts containing them, β-glycyrrhetinic acid and salts or derivatives thereof (stearyl glycyrrhetate, 3-stearoyloxyglycyrrhetic acid or glycyrrhetinic acid monoglucuronide) and also plants containing them (e.g., Glycyrrhiza glabra), oleanolic acid and salts thereof, ursolic acid and salts thereof, boswellic acid and salts thereof, betulinic acid and salts thereof, an extract of Paeonia suffruticosa and/or lactiflora, an extract of Laminaria saccharina, extracts of Centella asiatica, Canola oil, bisabolol, the phosphoric diester of vitamin E and C, for instance Sepivital EPC® from SEPPIC, camomile extracts, allantoin, omega-3 unsaturated oils such as musk rose oil, blackcurrant oil, Ecchium oil, fish oil or beauty-leaf oil, plankton extracts, capryloyl glycine, a mixture of water lily blossom extract and of palmitoylproline, such as the product marketed under the trademark Seppicalm VG® by SEPPIC, an extract of Boswellia serrata, an extract of Centipeda cunninghami, such as the product marketed under the trademark Cehami PF® by TRI-K Industries, an extract of sunflower seeds, in particular Hélioxine® from Silab, an extract of Linum usitatissimum seeds, for instance Sensiline® from Silab, tocotrienols, piperonal, an extract of Epilobium angustifolium, such as the product marketed under the trademark Canadian Willowherb Extract by Fytokem Products, Aloe vera, phytosterols, cornflower water, rose water, an extract of mint, in particular of mint leaves, for instance Calmiskin® from Silab, anise derivatives, filamentous bacteria, for instance Vitreoscilla filiformis as described in EP,761,204 and marketed by Chimex under the trademark Mexoryl SBG®, an extract of rose petals, for instance Rose Flower Herbasol® extract by Cosmetochem, shea butter, a mixture of the waxy fraction of barley seeds obtained by supercritical CO₂, of shea butter and of argan oil, for instance Stimu-tex AS® from Pentapharm, alkaline-earth metal salts, especially of strontium, a fermented extract of Alteromonas marketed under the trademark Abyssine® by Atrium Biotechnologies; spring water from the Vichy basin, such as waters originating from the Célestins, Chomel, Grande-Grille, Hopital, Lucas and Parc sources, and preferably water from the Lucas source; an extract of Eperua falcata bark, such as the product marketed by Cognis under the trademark Eperuline®; an extract of Paeonia suffruticosa root, such as the product marketed by Ichimaru Pharcos under the trademark Botanpi Liquid B®; and mixtures thereof.

### Hydrophilic Gelling Agent

The makeup composition may include a hydrophilic gelling agent. In some embodiments, a hydrophilic gelling agent may be present in the composition in a total amount of 0-0.2%, 0-0.1%, 0-0.05%, or 0-0.02% by weight of the composition. In some embodiments, the composition may be substantially free of a hydrophilic gelling agent. In some embodiments, the composition may be free of a hydrophilic gelling agent.

Hydrophilic gelling agents that may be mentioned in particular include water-soluble or water-dispersible thickening polymers. These polymers may be chosen especially from:
modified or unmodified carboxyvinyl polymers, such as the products sold under the name Carbopol (CTFA name: Carbomer) by the company Goodrich; polyacrylates
polymethacrylates such as the products sold under the names Lubrajel and Norgel by the company Guardian or under the name Hispagel by the company Hispano Chimica;
polyacrylamides; optionally crosslinked and/or neutralized 2-acrylamido-2-methylpropanesulfonic acid polymers and copolymers, for instance the poly(2-acrylamido-2-methylpropanesulfonic acid) sold by the company Clariant under the name Hostacerin AMPS (CTFA name: ammonium polyacryldimethyltauramide); crosslinked anionic copolymers of acrylamide and of AMPS, which are in the form of a W/O emulsion, such as those sold under the name Sepigel 305 (CTFA name: Polyacrylamide/C13-14 isoparaffin/Laureth-7) and under the name Simulgel 600 (CTFA name: Acrylamide/Sodium acryloyldimethyltaurate copolymer/Isohexadecane/Polysorbate 80) by the company SEPPIC;
polysaccharide biopolymers, for instance xanthan gum, guar gum, carob gum, acacia gum, scleroglucans, chitin and chitosan derivatives, carrageenans, gellans, alginates, celluloses such as microcrystalline cellulose, carboxymethylcellulose, hydroxymethylcellulose and hydroxypropylcellulose; and mixtures thereof.

### Fillers

The composition may include a filler. The composition may be substantially free of a filler. In some embodiments, a filler may be present in the composition in a total amount of 0-0.2%, 0-0.1%, 0-0.05%, or 0-0.02% by weight of the composition. The composition may be free of a filler.

The filler may be a mineral filler. The composition may be substantially free of a mineral filler. The composition may be free of a mineral filler. Non-limiting examples of mineral fillers include natural or synthetic mica, talc, kaolin, natural or synthetic sericite, silica, hydroxyapatite, boron nitride, calcium carbonate, hollow silica microspheres (Silica beads from Maprecos), glass or ceramic microcapsules; composites of silica and titanium dioxide, such as the TSG series sold by Nippon Sheet Glass, and mixtures thereof.

The filler may be an organic filler. The composition may be substantially free of an organic filler. The composition may be free of an organic filler. Non-limiting examples of organic fillers include polyamide powders (Nylon® Orgasol from Atochem), poly-β-alanine powder and polyethylene powder, polytetrafluoroethylene (Teflon®) powder, lauroyllysine, tetrafluoroethylene polymer powders, and spherical powders of crosslinked elastomeric organopolysiloxane, silicone resin microbeads, and/or polyurethane powders.

### Synthetic Peptides

In some embodiments, the composition may include a synthetic peptide. The synthetic peptide may be present in a total amount less than 0.1%, less than 0.01%, less than 0.001%, or less than 0.0001% by weight of the composition. In some embodiments, such synthetic peptides include the biopeptide CL, or various palmitoyl peptides, such as palmitoyl tripeptide-1 or palmitoyl tetrapeptide-7. In some embodiments, the composition may be substantially free of a synthetic peptide. In some embodiments, the composition may be free of a synthetic peptide.

### Additives and Other Components

In addition to the materials disclosed above, in some embodiments, the composition may be substantially free of all other materials. In addition to the materials disclosed above, in some embodiments, the composition may include no more than 0.25%, no more than 0.2%, no more than 0.15%, no more than 0.1%, or no more than 0.05% by weight of all other materials combined. In addition to the materials disclosed above, in some embodiments, the composition may be free of all other materials.

The makeup composition should be free of supramolecular polymers. As used herein, the term “supramolecular polymer” means a polymer chain or network formed from the assembly of a (co) polymer with at least one other identical or different (co) polymer, each assembly comprising at least one pair of identical or different paired junction groups. Such supramolecular polymers may be a supramolecular polymer as described in U.S. Pat. Nos. 9,000,051, 9,561,390, or FR 2938758.

In various aspects, a method for providing improved skin aesthetics may be provided. The method may include providing a first makeup composition as disclosed herein and applying the first makeup composition to a user's face. The method may include applying a second makeup composition over the first makeup composition. Alternatively, the method may include applying a second makeup composition to the user's face prior to the first makeup composition being applied, where the first makeup composition is applied over the second makeup composition. The method may include removing the first makeup composition after a period of time, the period of time being a time between 1 hour and 48 hours.

### Example 1

A base composition was used to create several formulas-ones with no silica (Comp. 1), lower silica (Comp. 2), and higher silica (Comp. 3) against compositions with silica within the claimed range (Ex. 1). The amount C15-C19 alkane was adjusted such that the total amount of silica+C15-C19 alkane remained constant. Specifically, formulas according to those described in Table 1 were formulated.

TABLE 1
Silica Level Comparative Formulations

| Material | Comp. 1 (wt %) | Comp. 2 (wt %) | Comp. 3 (wt %) | Ex. 1 (wt %) |
|---|---|---|---|---|
| Silica | 0 | 5 | 25 | 10-20 |
| C15-C19 Alkane | 30 | 25 | 5 | 10-20 |
| Base Composition | 70 | 70 | 70 | 70 |
| Candelilla Wax | 4 | 4 | 4 | 4 |
| Silicone Emulsifier | ~2.5 | ~2.5 | ~2.5 | ~2.5 |
| Silicone Oil | 4 | 4 | 4 | 4 |
| Other Cosmetic | ~21 | ~21 | ~21 | ~21 |
| Ingredients | | | | |
| Water | q.s. | q.s. | q.s. | q.s. |

Comparative formulas 1 and 2 (Comp. 1 and Comp. 2) were unstable (from a shelf stability standpoint), showing wax separation at 45° C. after 1 week. Comparative formula 3 (Comp. 3) was a paste, rather than a solid emulsion, and it did not blend or apply evenly on the skin, nor was it transparent on the skin (leaving a white cast on the skin after application). Only the exemplary products were shelf-stable, transparent on skin, solid emulsions.

### Example 2

Compositions according to those described in Table 2 have been created. Generally, oil-phase components are combined, water-phase components are combined separately and added to the oil-phase, followed by addition of fragrances.

TABLE 2

| Material | Ex. 2 (wt %) |
|---|---|
| Water | 35-40 |
| Silicas | 12-17 |
| C15-C19 Alkane | 12-17 |
| Candelilla Wax | 2-5 |
| Silicone Emulsifiers | 2-5 |
| Silicone Oils | 2-5 |
| Non-Silicone Emulsifiers | 1-2 |
| Non-Alkane Hydrocarbon Based Oils | 2-5 |
| Fragrance | <1 |
| Polysaccharides | 1-3 |
| Polyols | 10-15 |
| Pigments | 1-3 |
| Ceramides | <0.2 |
| Conditioning Agent | 1-2 |
| Antioxidants | <0.5 |
| Phenoxyethanol | <1 |
| All Other Cosmetic Ingredients | <0.2 |

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A makeup composition, comprising:
an aqueous discrete phase;
a fatty continuous phase including an alkane, the alkane present in an amount of 5-30% by weight of the makeup composition;
silica in an amount of 10% to 20% by weight of the makeup composition;
wherein the makeup composition is free of UV-absorbing composite particles;
wherein the makeup composition is free of supramolecular polymers; and
wherein the makeup composition is a solid emulsion.

2. The makeup composition of claim 1, wherein the alkane comprises a C15-C19 alkane.

3. The makeup composition of claim 1, wherein the aqueous discrete phase contains more water, by weight of the makeup composition, than the amount of both silica and alkane, separately.

4. The makeup composition of claim 3, wherein a total amount of water by weight of the makeup composition is greater than a total combined amount of the silica and the alkane combined by weight of the makeup composition.

5. The makeup composition of claim 3, wherein a total amount of water is 30% to 40% by weight of the makeup composition.

6. The makeup composition of claim 3, further comprising a silicone emulsifier.

7. The makeup composition of claim 6, further comprising a silicone oil.

8. The makeup composition of claim 7, further comprising a wax in a total amount of 2% to 5% by weight of the makeup composition.

9. The makeup composition of claim 1, further comprising a pigment.

10. The makeup composition of claim 1, further comprising a polyol.

11. The makeup composition of claim 1, further comprising cellulose and/or a cellulose derivative.

12. The makeup composition of claim 1, wherein the makeup composition is free of organic UV filters.

13. A method for providing improved skin aesthetics, comprising:
providing a first makeup composition according to claim 1; and
applying the first makeup composition to a user's face.

14. The method of claim 13, further comprising applying a second makeup composition over the first makeup composition.

15. The method of claim 13, further comprising applying a second makeup composition to the user's face prior to the first makeup composition being applied, where the first makeup composition is applied over the second makeup composition.

16. The method of claim 13, further comprising removing the first makeup composition after a period of time, the period of time being a time between 1 hour and 48 hours.
